(19) United States
(12) Patent Application Publication
(10) Pub. No.: US 2016/0326070 A1
(43) Pub. Date: Nov. 10, 2016

(54) DILUTING ALKANE OXYDEHYDROGENATION REACTANTS WITH CARBON DIOXIDE

(71) Applicant: LINDE AKTIENGESELLSCHAFT, München (DE)

(72) Inventors: Florian Winkler, München (DE); Andreas Meiswinkel, Prien (DE); Desislava Tota, München (DE)

(21) Appl. No.: 15/110,186

(22) PCT Filed: Jan. 22, 2015
(86) PCT No.: PCT/EP2015/000122

§ 371 Date: Jul. 7, 2016

(30) Foreign Application Priority Data
Jan. 30, 2014 (EP) 14000346.8

(51) Int. Cl. C07C 5/48

(52) U.S. Cl. CPC C07C 5/48

(57) ABSTRACT

A process for oxydehydrogenating an alkane to a corresponding alkene, particularly ethane to ethylene, wherein a feed comprising the alkane, an oxygen-containing oxidizing agent and a diluent comprising CO₂ are provided to a reactor. Oxidative dehydrogenation with oxygen takes place in the reactor in the presence of a catalyst to convert the alkane to a product stream which includes the corresponding alkene. The oxygen used as the oxidizing agent may be supplied in stoichiometric amount or in stoichiometric excess.

The invention relates to a process for oxydehydrogenating (ODH) an alkane to the corresponding alkene, particularly ethane to ethylene, wherein a feed comprising at least an alkane and an oxygen-containing oxidizing agent is provided in a reactor and an oxydehydrogenation with oxygen takes place in the reactor in the presence of a catalyst to convert the alkane to a gaseous product stream which includes the corresponding alkene.

The oxidative dehydrogenation (oxydehydrogenation) of ethane to ethylene is known from the prior art.

The oxidative dehydrogenation of an alkane to the corresponding alkene, particularly ethane to ethylene, is a strongly exothermic process. Particularly the formation of by-products by overoxidation to CO and CO₂ releases a disproportionately large amount of heat. A significant increase in the temperature due to the reaction serves in turn to promote the overoxidation and thus leads to a destruction of valuable raw materials and, in particular, to increased formation of CO and of the climate killer CO₂.

Oxygen is generally used as oxidizing agent in the oxidative dehydrogenation, although the use of CO₂ as oxidizing agent is also known, for example from the printed publications L. Liu, H. Jiang, H. Liu, H. Li, “Chapter 7—Recent Advances on the Catalyst for Activation of CO2 in Several Typical Processes” in: New and Future Developments in Catalysis, Elsevier, 189-222 (2013), US2010087615A1, CA2561986A1, U.S. Pat. No. 2,604,495 and U.S. Pat. No. 6,037,511.

In order to control the exothermicity of oxydhydrogenation and not to exceed explosion limits, therefore, the feed (alkane and oxidizing agent, e.g. oxygen) is typically diluted. It is generally nitrogen and/or steam which are used for this as an inert diluent in industrial practice, as described for example in the printed publications WO2010115099A1, WO2010115108A1, US2005085678A1, US2001025129 A1 and U.S. Pat. No. 4,899,003A.

Dilutions of this kind, however, lead to problems in the fractionation part of such a plant. Water can be separated off by condensation, while the removal of nitrogen typically requires low temperatures, i.e. the provision of appropriate cooling power and also of the necessary apparatus for this (e.g. cooling circuit, distillation). Water, by contrast, can simply be separated off at moderate temperatures. The energy released can to a certain degree be reused for water vaporization and steam generation. However, the steam thus generated is generally insufficient to devise an adequate amount of diluent, so additional diluent has to be resorted to and/or, alternatively, additional energy has to be provided at the very least.

Against this background, therefore, the present invention has for its object to provide a process for the ODH of alkanes which is less demanding by way of apparatus requirements.

This object is achieved by a process having the features of Claim 1.

In said process, a diluent comprising CO₂ at least is provided, in particular for controlling the exotherm, as a constituent part of the feed, while the oxygen for partially oxidizing the alkane (particularly ethane) as per the equation (exemplified for ethane) 2C₂H₆+O₂->2C₂H₄+2H₂O

is further provided at an at least stoichiometric or else superstoichiometric amount relative to the alkane/ethane feed. CO₂ may also be formed in said process as a result of further oxidation of the alkene/ethylene in secondary reactions to CO and CO₂ and of the CO. This at least stoichiometric/superstoichiometric admixture of oxygen stops the CO₂ formed in the ODH from acting as an oxidizing agent. On the contrary, the CO₂ in this invention acts as a diluent and if it takes part in the reaction as an oxidizing agent it does so only to a very minor extent.

In one preferred embodiment of the invention, the CO₂ formed during the reaction by complete oxidation is separated off and recycled into the reactor as dilution medium in order to dilute the feed stream for better and safer reaction control. The diluent preferably consists completely or at least majorly of CO₂.

A disadvantage, which is consciously incurred, is the additional cost and inconvenience of providing oxygen and/or oxygen-enriched air. However, a distinctly lower level of cost and inconvenience is advantageously incurred as a result in the fractionation part of the process and/or processing plant, since significantly less, if any, N₂ now has to be separated from the product stream. Furthermore, the irrespectively required CO₂ removal can be used for the CO₂ recycle.

Preferably, in the case of an isothermally operated reactor, the proportion of the overall feed stream which is attributable to the diluent is in the range from 5% by volume to 90% by volume, preferably from 25% by volume to 75% by volume and more preferably from 40% by volume to 60% by volume.

Further preferably, in the case of an adiabatically operated reactor, the proportion of the overall feed stream which is attributable to the diluent is in the range from 50% by volume to 95% by volume, preferably from 60% by volume to 90% by volume and more preferably from 70% by volume to 85% by volume.

Preferably, the diluent comprises from 10% to 100% by volume of CO₂, preferably from 20% to 100% by volume of CO₂ and more preferably from 40% to 100% by volume of CO₂, the balance in each case, if present, consisting of H₂O and/or N₂, or including these components. H₂O and N₂ may here be used in any desired ratio relative to each other. When H₂O and/or N₂ are admixed, it is advisable to put the upper limit for the CO₂ in the diluent at particularly 20% by volume, preferably 40% by volume and more preferably 80% by volume.

It is preferably further provided that oxygen is used as the oxidizing agent in the ODH reaction at least stoichiometrically or in stoichiometric excess, wherein in particular the ratio of oxygen to freshly supplied alkane, in particular to freshly supplied ethane, in the feed is in the range of 0.50-1.1 (with the units: mol of O₂/mol of ethane), preferably of 0.53-1 (mol of O₂/mol of ethane) and more preferably in the range of 0.55-0.9 (mol of O₂/mol of ethane). This holds not only for an isothermal reactor but also for an adiabatically operated reactor.

An ethane recycle is further operated in a ratio (relative to the fresh ethane) of from 1/1 to 4/1 (the ethane recycle ratio is thus defined as ratio of recycle ethane to fresh ethane), preferably from 1/2 to 3/1 (recycle ethane/fresh ethane) and more preferably from 0 to 2/1 (recycle ethane/fresh ethane). Fresh ethane/alkane is thus herein to be understood as referring to ethane/alkane at the time of its first injection into the reactor. Recycle ethane, by contrast, is unconverted ethane being reinjected.

It is preferably further provided that CO₂ present in the product stream be separated off and recycled as diluent into the reactor. The CO₂ in the product stream is the CO₂ formed in the course of the oxidative dehydrogenation plus diluent that has passed through the reactor.

Advantageously, pure oxygen is provided as an oxidizing agent for the partial oxidation together with the CO₂ recycle of the present invention. The oxygen may be provided using an air separator or else via a pressure swing adsorption (PSA) plant. CO₂ is generally by-produced in the ODH reaction itself and may be injected from the outside as needed at the start of the process and/or to start up the plant.

The CO₂ is further readily removable from the product stream in a scrub (e.g. Rectisol or amine scrub) and recyclable into the process.

The use of such a CO₂ recycle while nitrogen is absent and/or a reduced nitrogen fraction is used reduces the separation requirements in the fractionation part. The CO formed and the residual oxygen O₂ are thus the only components left to separate from the product stream and the hydrocarbons present therein. The ejected heat of the ODH reaction may further be used with advantage to regenerate the CO₂ scrub.

It is preferably further provided in the process of the present invention that upstream of the removal of CO₂ from the product stream H₂O is removed or separated (in a separator in particular) from the product stream, and preferably the removed H₂O is converted into steam by means of a steam generator and is, in particular, recycled into the reactor as a diluent or used otherwise, for example as process steam.

It may additionally be provided according to the present invention that upstream of the removal of CO₂ from the product stream CO present in the product stream is converted into CO₂ via an oxidation, preferably a catalytic oxidation (also referred to as CATOX), wherein said oxidation is more particularly effected downstream of the removal of the H₂O from the product stream. CATOX may utilize for example catalysts such as platinum and palladium.

This oxidation thus advantageously ensures that the product stream comprises additional dilution medium which, at the subsequent CO₂ removal, may be recycled into the reactor. Such an optional CO removal may additionally minimize requirements in the fractionation part, since an explicit processing unit for removing CO can be omitted. A further advantage to an oxidation unit for converting CO into CO₂ is that the residual oxygen is minimized and/or fully converted. It is accordingly easier to comply with explosion limits in the fractionation part. Oxygen may further be harmful to scrubbing (depending on the scrubbing medium used). An oxidation unit thus also reduces the risk of scrubbing medium degeneration. Using CATOX further requires that the scrub and complete CO conversion be followed by the removal of mainly oxygen, which may likewise be recycled into the reactor.

In a further preferred embodiment, upstream of the removal of CO₂ from the product stream the product stream is compressed, wherein in particular the compression is effected downstream of the segregation of H₂O from the product stream, and wherein the compression is more particularly effected downstream or upstream of the (specifically catalytic) oxidation for conversion of CO to CO₂ in the gaseous product stream.

In a further preferred embodiment, upstream of the removal of CO₂ from the product stream a further removal of H₂O from the product stream is effected, more particularly downstream of the compression of the CO₂ and also more particularly downstream of the aforementioned optional catalytic oxidation. H₂O which has been removed or separated off (by means of a separator for example) may again be converted into steam (by means of a steam generator) and, in particular, be recycled into the reactor or used otherwise, for example as process steam.

It may further be provided that downstream of the removal of CO₂ from the product stream the product stream is compressed again or for the first time (see hereinbelow).

Preferably, downstream of the compression of the product stream at a point downstream of the removal of CO₂ from the product stream, O₂ and also, in particular, CO and/or N₂ (depending on the oxidizing agent used) are removed from the product stream, wherein in particular O₂ and also, in particular, CO are recycled into the reactor. Nitrogen, which is obtained when air or oxygen-enriched air is used as oxidizing agent, is preferably not recycled into the reactor.

In a further preferred embodiment, downstream of the compression of the product stream at a point downstream of the removal of CO₂ from the product stream, in particular downstream of the removal of O₂ and also, in particular, CO and/or N₂ from the product stream, the alkene, particularly ethylene, is separated from alkane, particularly ethane in the product stream, wherein in particular the alkane is recycled into the reactor.

As already mentioned above, the product stream from the reactor may be compressed either twice/via two compressors or merely once/via one compressor. In one version of the invention, the gaseous product stream is compressed before CO₂ is removed from the product stream (see above). In this case, it is possible to compress to the final pressure needed to separate the alkane from alkene. In an ODH of ethane to ethylene, the corresponding column is also known as a C2 splitter. Compression before the removal of O₂, CO and/or N₂ from the product stream (known as a demethanizer because C₁ is removed) can then be omitted.

In a two-stage compression, by contrast, the gaseous product stream is preferably compressed in the first stage to the extent needed to separate/scrub CO₂ out of the product stream. In the second stage, the product stream is then further compressed to the required pressure for separating the alkane from the alkene in the product stream or to the required pressure for the C2 splitter.

When the ODH reaction takes place at a pressure which is already sufficient to separate CO₂ out of the product stream, the first compression/compressor can be omitted and the second compression/compressor described becomes mandatory.

When a CO₂ recycle according to the present invention is used, the result is accordingly a distinct reduction in the gas load for compression in the scenario where no compression is needed before CO₂ removal or a two-stage compression is provided.

Preferably, pure oxygen is used as oxidizing agent in the process of the present invention. Alternatively, however, it is also possible to use air or oxygen-enriched air as oxidizing agent. The nitrogen is then preferably separated from the product stream in a rectification column and preferably not recycled into the reactor.

Further features and advantages of the invention will now be elucidated in the figure description of exemplary embodiments of the invention by reference to the figures. In the drawing

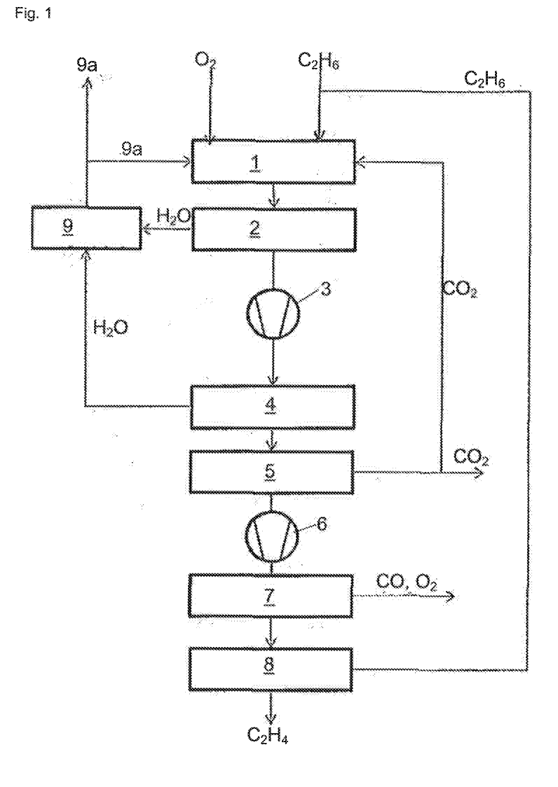

FIG. 1 shows a schematic depiction of a first embodiment of the inventive process;

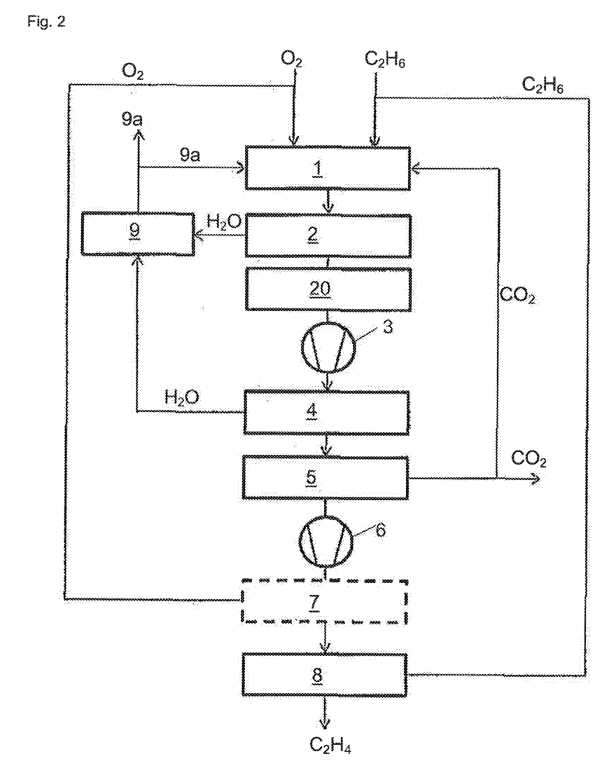

FIG. 2 shows a schematic depiction of a second embodiment of the inventive process;

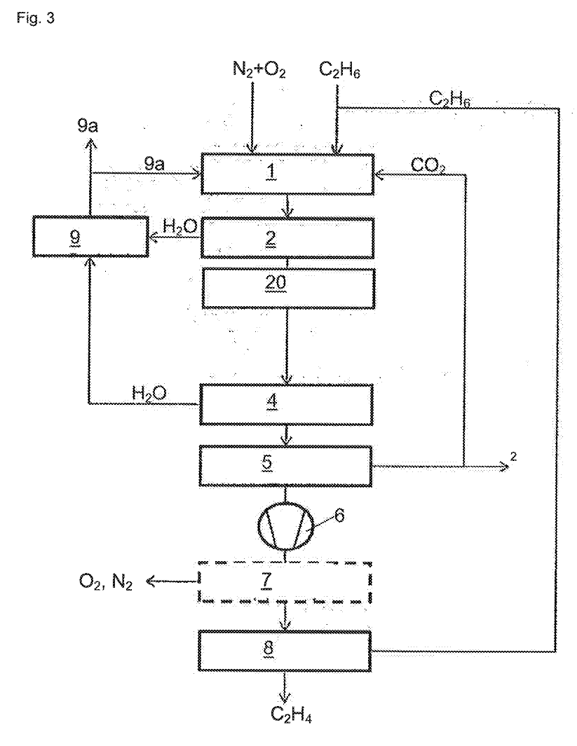

FIG. 3 shows a schematic depiction of a third embodiment of the inventive process;

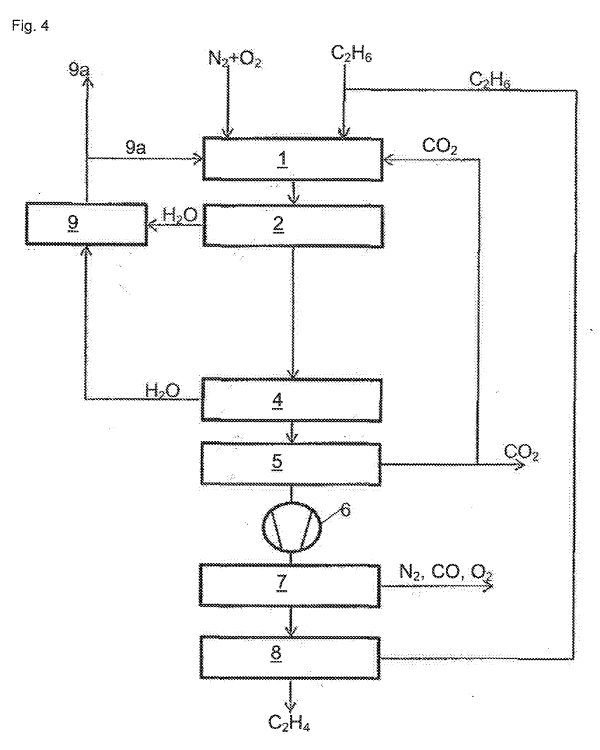

FIG. 4 shows a schematic depiction of a fourth embodiment of the inventive process; and

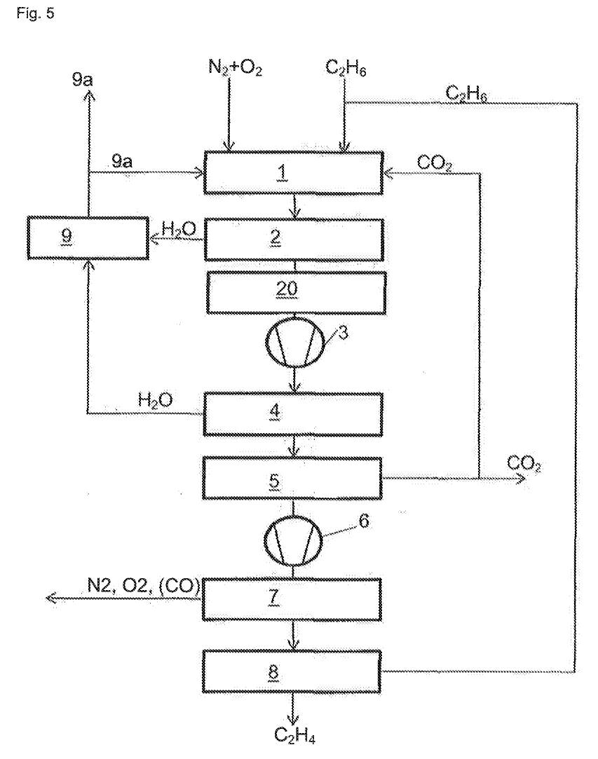

FIG. 5 shows a schematic depiction of a fifth embodiment of the inventive process;

FIG. 1 shows a first embodiment of the inventive process wherein ethane is reacted with oxygen in a reactor 1 in an oxidative dehydrogenation to ethylene, the resultant gaseous product stream P comprising ethane, CO, CO₂, H₂O and oxygen as well as ethylene. The invention is described herein with reference to the ODH of ethane. Other alkanes are oxydehydrogenatable in a similar manner. The ODH takes place in reactor 1 at a pressure which is, for example, in the range from 0.5 bar to 25 bar, preferably from 1 bar to 15 bar and more preferably from 3 bar to 10 bar, in the presence of a suitable catalyst (see also below).

The reactor effluent, i.e. the product stream P generated in the reactor, is then introduced into a separator 2 to separate H₂O from the product stream P. The removed H₂O may optionally be vaporized in a steam generator 9 and recycled into reactor 1 or be used otherwise. The steam generator may utilize ODH waste heat for steam generation, for example.

The gaseous, dried product stream P is passed from the separator 2 into a compressor 3 and compressed and then reintroduced into a separator 4 to remove H₂O from the product stream P. Removed H₂O may again be sent to the steam generator 9 and be recycled into the reactor 1, or used otherwise, in the form of steam.

CO₂ in product stream P is subsequently removed from product stream P, by scrubbing for example, and is in accordance with the present invention recycled as diluent into the reactor 1, or discarded.

After the CO₂ has been removed, the product stream P is recompressed, say to a pressure in the range from 25 bar to 35 bar, and then has CO and any oxygen still present removed from it by distillation in a column 7 (known as a demethanizer because C₁, i.e. in particular O₂ and also any N₂ are removed as well as CO) and are more particularly recycled into the reactor 1. The product stream P is subsequently introduced into a C2 splitter 8 where ethane present in product stream P is separated from ethylene present in product stream P, and the ethane is recycled into the reactor 1.

FIG. 2 shows a version of the process according to FIG. 1 wherein, in contradistinction to FIG. 1, downstream of H2O removal 2 and downstream of product stream P compression 3 a catalytic oxidation 20 is carried out to convert the CO in product stream P into CO₂ which is additionally removed from product stream P in scrub 5 and recycled as diluent into reactor 1. This further makes it possible to omit the removal 7 of O₂ and CO from the product stream, as indicated in FIG. 2, particularly when O₂ and CO are removed in the CATOX to such an extent that they are no longer disruptive in the ethylene product and also in the fractionation part.

FIG. 3 shows a further version of the inventive process wherein, in contradistinction to FIGS. 1 and/or 2, no compression is provided between the two water removals 2 and 4, but merely said catalytic oxidation 20 where CO in product stream P is converted into CO₂ which is additionally removed from product stream P in scrub 5 and recycled into reactor 1. It is further provided in this version that air or oxygen-enriched air is introduced as oxidizing agent into reactor 1 and N₂ is removed 7 preferably cryogenically in a rectification column but is not returned into reactor 1. Oxygen-enriched air may be provided via pressure swing adsorption (PSA) in a conventional manner.

FIG. 4 shows a further version of the inventive process wherein, in contradistinction to FIG. 3, no catalytic oxidation 20 is carried out. Incompletely converted CO as well as O₂ and N₂ is separated from product stream P at 7, after compression 6, before the latter is introduced into the C₂ splitter 8.

FIG. 5 finally shows a further embodiment of the inventive process wherein, in contradistinction to FIG. 3, CATOX 20 is followed on its downstream side by a compression of product stream P in order to enhance the degree of CO₂ scrub-out in the subsequent scrub 5 where CO₂ is removed from product stream P and recycled into reactor 1.

The exemplary embodiments described are performable with any oxydehydrogenating catalyst that is stable under the reaction conditions. Preferably, however, the exemplary embodiments utilize for the ODH reaction a metal oxide catalyst that includes the elements Mo, V, Te and Nb.

This may be, for example, a catalyst of the MoVₐTe_{b}Nb_{c}Oₓ class, where a is preferably from 0.05 to 0.4, b is preferably from 0.02 to 0.2 and c is preferably from 0.05 to 0.3. In the above formula MoVₐTe_{b}Nb_{c}Oₓ, x is the molar number of the oxygen which binds to the metal atoms of the catalyst, and it follows from the relative amount and valence of the metal elements. This can also be expressed by the formula MoVₐTe_{b}Nb_{c}Oₓ, where s, p, q and r are the oxidation states of Mo, V, Te and Mb, respectively, subject to the proviso that 2·x=s+p·a+b·q+c·r. Mo may be present not only in the oxidation state +5 but also in the oxidation state +6. V may be present in the oxidation states +4 and +5, depending on the position in the crystal. Niobium is present in the oxidation state +5. Tellurium is present in the oxidation stage +4.

Reactor 1 as described in the embodiments may further be both isothermal and adiabatic in construction/operation.

When reactor 1 is used in the form of an isothermal reactor, for example in the form of a molten-salt reactor, the following parameters for example may be used as process data:
from 0.5 bar to 25 bar, preferably from 1 bar to 15 bar and more preferably from 3 bar to 10 bar for the pressure in reactor facility 1,
from 250° C. to 650° C., preferably from 280° C. to 550° C. and more preferably from 350° C. to 480° C. for the temperature in reactor facility 1.
Feed compositions (feed stream E):
   preferably from 7% by volume to 86% by volume of ethane, from 1% by volume to 50% by volume of O2, from 1% by volume to 90% by volume of CO₂, balance H₂O and/or N₂, preferably from 16% by volume to 66% by volume of ethane, from 3% by volume to 38% by volume of 02, from 5% by volume to 75% by volume of CO₂, balance H₂O and/or N₂, more preferably from 21% by volume to 55% by volume of ethane, from 6% by volume to 30% by volume of O₂, from 16% by volume to 60% by volume of CO₂, balance H₂O and/or N₂.
Weight hourly space velocity (WHSV) is preferably in the range from 1.0 kg to 40 kg of C₂H₆/h/kg of cat, preferably in the range from 2 kg to 25 kg C₂H₆/h/kg of cat and more preferably in the range from 5 kg to 20 kg C₂H₆/h/kg of cat.

When reactor 1 is used in the form of an adiabatic reactor, the following parameters for example can be used as process data:
from 0.5 bar to 25 bar, preferably from 1 bar to 15 bar and very preferably from 3 bar to 10 bar for the pressure in reactor facility 1,
from 250° C. to 650° C., preferably from 280° C. to 550° C. and very preferably from 350° C. to 480° C. for the temperature in reactor facility 1.
Feed compositions (feed stream E):
   preferably from 3% by volume to 45% by volume of ethane, from 1% by volume to 26% by volume of O₂, from 5% by volume to 95% by volume of CO₂, balance H₂O and/or N₂, preferably from 6% by volume to 35% by volume of ethane, from 2% by volume to 20% by volume of O₂, from 12% by volume to 90% by volume CO₂, balance H₂O and/or N₂, more preferably from 8% by volume to 25% by volume of ethane, from 3% by volume to 15% by volume of O₂, from 28% by volume to 85% by volume of CO₂, balance H₂O and/or N₂.
Weight hourly space velocity (WHSV) is preferably in the range from 2.0 kg to 50 kg of C₂H₆/h/kg of cat, preferably in the range from 5 kg to 30 kg of C₂H₆/h/kg of cat, and more preferably in the range from 10 kg to 25 kg C₂H₆/h/kg of cat.
The proportion of an inert material added to the catalyst may be up to 90% by volume based on the fixed bed, preferably it is from 30% by volume to 85% by volume and more preferably from 50% by volume to 75% by volume, all based on the fixed bed. A second or further fixed bed may optionally follow on the downstream side, and it may be implemented without inert material.

The above-described process of the present invention simplifies the apparatus requirements because the fractionation part has lower requirements. A demethanizer (N₂ and CO removal) 7 may be eschewed, if desired.

CO₂ removal from the product is required in any event. Using and implementing a CO2 recycle merely necessitates a higher designed capacity for the CO₂ scrub, in favour of savings in relation to the cryogenic removal and recycling of inerts such as N₂ in the fractionation part and/or the thermal provision of steam as diluent. The CATOX system 20 further provides a simple way to convert CO into CO₂ and provide additional diluent.

It is further possible to reduce the gas load in the above-described compression stages.

Gas purification further turns out to be energy efficient to operate. Scrub 5 is generally operated at temperatures >40° C., while the N₂/C₂₊ separation requires additional cooling below −150° C. at about 13 bar.

The scrubbing medium may advantageously be regenerated using rejected heat from reactor 1.

CATOX 20 as described further brings about a synergetic effect. CATOX 20 increases the CO₂ fraction for the inert gas recycle (CO₂), while the conversion of CO into CO₂ further makes it possible to dispense with a rectification column for CO/C₂ separation.

Additionally removing and reducing the oxygen further makes it possible to reduce the explosion risk and brings about a saving in the gas clean-up.

Additionally removing and reducing the oxygen further results in a reduced scrubbing medium degeneration on using, for example, amine scrubs 5. An oxygen stream is easy to separate off and recycle into reaction 1.

Steam is generally used to minimize the N₂ recycle. In the present case, steam 9a can be completely eschewed. Steam generation 9 can be effected using the rejected heat from reactor 1 and by cooling the product stream P (about 400° C.). The eschewal of steam 9a as diluent medium has the following advantages: the steam 9a can be exported, the steam 9a can be used as heat transfer medium in the fractionation part, and vaporizers can be dispensed with completely.

Condensing out the steam further results in increased O₂ content. The eschewal of steam 9a as diluent is therefore advantageous in reducing the risk of reaching explosion limits.

List of reference signs

| | |
|---|---|
| 1 | reactor |
| 2 | separator |
| 3 | compressor |
| 44 | separator |
| 5 | CO₂ removal (e.g. scrub) |
| 6 | compressor |
| 7 | removal of O₂, CO and/or N₂ |
| 8 | C2 splitter (separation of ethane and ethylene) |
| 9 | steam generation |
| 9a | steam or, to be more precise, process steam |
| 20 | CATOX |
| P | product stream |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A process for oxydehydrogenating an alkane to a corresponding alkene comprising:
providing a feed of at least an alkane and oxygen as oxidizing agent to a reactor;
converting the alkane to a product stream which includes the corresponding alkene by oxydehydrogenation of the alkane with oxygen in the reactor in the presence of a catalyst, wherein
the feed further includes a diluent comprising CO₂ as an oxidizing agent.

17. The process according to claim 16, wherein the alkane is ethane and the corresponding alkene is ethylene.

18. The process according to claim 16, wherein the reactor is operated isothermally and the proportion of diluent in the feed is from 5% by volume to 90% by volume.

19. The process according to claim 18, wherein the proportion of diluent in the feed is from 25% by volume to 75% by volume.

20. The process according to claim 19, wherein the proportion of diluent in the feed is from 40% by volume to 60% by volume.

21. The process according to claim 16, wherein the reactor is operated adiabatically and the proportion of diluent in the feed is from 50% by volume to 95% by volume.

22. The process according to claim 21, wherein the proportion of diluent in the feed is from 60% by volume to 90% by volume.

23. The process according to claim 22, wherein the proportion of the diluent in the feed is from 70% by volume to 85% by volume.

24. The process according to claim 16, wherein the diluent includes 10% by volume to 100% by volume of CO₂ and the balance consists of H₂O, N₂ or a mixture of H₂O and N₂.

25. The process according to claim 24, wherein the diluent includes 20% by volume to 100% by volume of CO₂.

26. The process according to claim 25, wherein the diluent includes 40% by volume to 100% by volume of CO₂.

27. The process according to claim 24, wherein the diluent includes 10% by volume to 20% by volume of CO₂ and the balance consists of a mixture of H₂O and N₂.

28. The process according to claim 24, wherein the diluent includes from 20% by volume to 40% by volume of CO₂, and the balance consists of a mixture of H₂O and N₂.

29. The process according to claim 24, wherein the diluent includes from 40% by volume to 80% by volume of CO₂, and the balance consists of a mixture of H₂O and N₂.

30. The process according to claim 16, wherein the alkane in the feed is comprised of fresh alkane and alkane recycled that has not been converted in the oxydehydrogenating process.

31. The process according to claim 30, where the ratio of recycled alkane to fresh alkane in the feed is from 1:1 to 4:1.

32. The process according to claim 31, where the ratio of recycled alkane to fresh alkane in the feed is from 1:2 to 3:1.

33. The process according to claim 32, where the ratio of recycled alkane to fresh alkane in the feed is from 1:2 to 2:1.

34. The process according to claim 30, wherein the diluent includes oxygen in a stoichiometric amount or a stoichiometric access.

35. The process according to claim 34, wherein the ratio of mol of oxygen to mol of fresh alkane in the feed is from 0.50 to 1.1.

36. The process according to claim 35, wherein the ratio of mol of oxygen to mol of fresh alkane in the feed is from 0.53 to 1.

37. The process according to claim 36, wherein the ratio of mol of oxygen to mol of fresh alkane in the feed is from 0.55 to 0.9.

38. The process according to claim 30, wherein the oxygen is pure oxygen.

39. The process according to claim 30, wherein the oxygen is provided as air or oxygen-enriched air.

40. The process according to claim 16, wherein the product stream includes CO₂ and the process further comprises
removing the CO₂ from the product stream; and
recycling the removed CO₂ to the reactor as at least part of the diluent.

41. The process according to claim 40, further comprising
removing H₂O from the product stream upstream of the step of removing CO₂ from the product stream;
converting the removed H₂O into steam; and
recycling the steam to the reactor or using the steam as process steam.

42. The process according to claim 41, further comprising:
removing CO from the product stream upstream of step of removing CO₂ from the product stream and downstream of the step or removing H₂O from the product stream; and
converting the removed CO into CO₂ by oxidation.

43. The process according to claim 42, wherein the step of converting CO into CO₂ is carried out by catalytic oxidation.

44. The process according to claim 42, further comprising
compressing the product stream upstream of the step of removing CO₂ from the product stream, downstream of the step or removing H₂O, and upstream or downstream of the step of converting CO.

45. The process according to claim 44, further comprising
a second step of removing H2O from the product stream upstream of the step of removing CO₂ from the product stream, downstream of the step of compressing the product stream and downstream of the step of converting CO;
converting the removed H₂O into steam; and
recycling the steam to the reactor or using the steam as process steam.

46. The process according to claim 40, further comprising
compressing the product stream downstream of the step of removing CO₂ from the product stream.

47. The process according to claim 46, further comprising
removing CO and N₂ from the product stream downstream of the step of compressing the product stream; and
recycling the removed CO and N₂ into the reactor.

48. The process according to claim 47, further comprising
separating alkene from alkane downstream of the step of removing CO and N₂ from the product stream; and
recycling the separated alkane into the reactor.
